(19) United States
(12) Patent Application Publication
(10) Pub. No.: US 2018/0085525 A1
(43) Pub. Date: Mar. 29, 2018

(54) DRUG DELIVERY SYSTEM AND METHOD

(71) Applicant: Preciflex SA, Neuchâtel (CH)

(72) Inventors: Lucien VOUILLAMOZ, Feusisberg (CH); Michel BRUEHWILER, Newton, MA (US); Patrick SCHERRER, Geneva (CH)

(73) Assignee: Preciflex SA, Neuchâtel (CH)

(21) Appl. No.: 15/524,748

(22) PCT Filed: Nov. 6, 2015
(86) PCT No.: PCT/IB2015/002075

§ 371 Date: May 5, 2017

Related U.S. Application Data
(60) Provisional application No. 62/076,194, filed on Nov. 6, 2014.
(60) Provisional application No. 62/134,025, filed on Mar. 17, 2015.

(30) Foreign Application Priority Data
Oct. 5, 2015 (IB) PCT/IB2015/000177

(51) Int. Cl. A61M 5/178; A61M 5/315

(52) U.S. Cl. CPC A61M 5/1782; A61M 5/204; A61M 5/31556

(57) ABSTRACT

An insulin delivery system and method for dispensing a measured, user selected, amount of insulin, a derivate thereof, or another substance into an animal via a dosing module and a delivery module. The system includes a reusable dosing module. The reusable dosing module is adapted to extract a set volume of the insulin, a derivate thereof, or another substance and a set volume of the diluent from reservoir(s). The reservoir(s) optionally includes a vial, and transfers the set volume(s) to a single use, manually activated injector reservoir. The reusable injector carrier module is configured to house the single use manually activated injector reservoir and configured to provide a surface to stroke or calm the animal prior to injection.

### CROSS REFERENCE TO RELATED APPLICATIONS

This application claims the benefit of U.S. Provisional Application No. 62/076,194, filed Nov. 6, 2014 and U.S. Provisional Application 62/134,025, filed Mar. 17, 2015, both entitled DRUG DELIVERY SYSTEM AND METHOD FOR FELINES, as well as PCT/IB2015/001777, filed 5 Oct. 2015, entitled WEARABLE FLUID DISPENSING DEVICES, SYSTEMS AND METHODS RELATED THERETO, the content of the entirety of which is explicitly incorporated herein by reference and relied upon to define features for which protection may be sought hereby as it is believed that the entirety thereof contributes to solving the technical problem underlying the invention, some features that may be mentioned hereunder being of particular importance.

### COPYRIGHT & LEGAL NOTICE

A portion of the disclosure of this patent document contains material which is subject to copyright protection. The copyright owner has no objection to the facsimile reproduction by anyone of the patent document or the patent disclosure as it appears in the Patent and Trademark Office patent file or records, but otherwise reserves all copyright rights whatsoever. Further, no reference to third party patents or articles made herein is to be construed as an admission that the present invention is not entitled to antedate such material by virtue of prior invention.

### BACKGROUND OF THE INVENTION

This invention relates to fluid dispensers and in particular to dispensers for the measured delivery of insulin, a derivate thereof, or another substance, to, for example, a feline (which term is used herein for exemplary purposes, the invention being applicable to any other animal including human beings).

Prior devices that are capable of injecting insulin, a derivate thereof, or another substance in an animal upon a trigger event are cumbersome and bulky, requiring significant or dedicated space, lack accuracy (especially for doses under 1 insulin unit (UI) with a concentration of 40 UI/10 ml), presenting a certain danger when the needle is ready to perform the injection and therefore requiring a complicated process to avoid it, or are too costly for many users. What is needed is an insulin, a derivate thereof, or another substance dispenser that provides an injection of a protein or substance to an animal and yet remains user-friendly, secure, simple in process, reliable and highly compact.

### SUMMARY OF THE INVENTION

A fluid delivery system and method for dispensing a measured amount of fluid, such as insulin, a derivate thereof, into an animal is provided. The system includes a reusable dosing module. The reusable dosing module is adapted to extract and dilute a set volume of the fluid from a larger fluid vial with a solvent provided by a solvent vial. The dilution system transfers a fixed amount of the resulting mixture to the reservoir placed into a disposable, single use, manually activated injector. It has to be observed, that the volume of the injector reservoir is fixed, allowing the user to use always the same disposable injector type for a treatment, also the amount of insulin must varied from injection to injection. A diluent vial is adapted to provide a diluent. A reusable injector carrier is configured to house the single use manually activated injector and configured to provide a surface to stroke or calm the animal prior to injection. The carrier assembly is adapted to removably house the single use manually activated injector such that the system provides for proper dosing of the measured amount of insulin subcutaneously into an animal.

In a variant, the invention provides a method of dosing an animal with a variably adjustable amount of animal-specific drug, such as insulin. The method comprises the steps of: inserting the vial containing the insulin with a specific concentration and the vial containing the adapted diluent of that specific insulin into a dosing module; dialling a suitable dose of insulin which will be contained in the injector reservoir with a dose selection wheel and/or slider; dialling a suitable dose of insulin concentration of the vial containing the insulin to be diluted; pushing a button to realise the dilution of the insulin with the diluent and to transfer the resulting mixture so that the previously selected insulin dose is reached into the specific fixe volume of the injector reservoir; removing the disposable injector; placing the injector into the reusable injector carrier; removing the security bracelet from the injector, taking over the carrier and placing it at an injection site such that a needle hole guard touches the skin of the animal; pushing the injector head to 1) trigger needle insertion through the skin of the animal and 2) dispense the insulin, a derivate thereof, or another substance from the injector reservoir into the skin of the animal; 3) removing the needle from the skin and 4) removing the injector from the carrier and discarding it.

In yet another variant, a substance delivery system for dispensing a measured amount of insulin, a derivate thereof, or another substance into an animal is provided. The system includes a reusable dosing module. The reusable dosing module is adapted to extract a set volume of the insulin, a derivate thereof, or another substance from a larger reservoir upon selection of a volume using one or more dosing mechanisms. The larger reservoir is optionally a vial, and the method includes the transferring of a set volume to a single use, manually activated injector. A reusable injector carrier is configured to house the single use manually activated injector and configured to provide a surface to stroke or calm the animal prior to injection. The carrier assembly is adapted for removably housing the single use manually activated injector. The system provides for proper dosing of the measured amount of animal specific insulin, a derivate thereof, or another substance subcutaneously into an animal once selection is made using the dosing and/or concentration wheel and/or slider mechanism(s).

In yet a further aspect, the invention includes a method of dosing an animal with a variably adjustable amount of fluid with the dose being a therapeutically effective dose. The method includes inserting a glass vial and optionally a diluent vial into the dosing module to: pierce the septum of the glass vial and/or diluent vial, and establish a fluidic path from the glass vial to the internal dosing mechanism of glass vial and the internal fluidic path/mechanism of dosing module, and establish a fluidic path from the diluent vial to the internal dosing mechanism of diluent vial and the internal fluidic path/mechanism of dosing module; dialling a suitable dose of insulin, a derivate thereof, or another substance with one or more of dose set wheels and/or sliders, checking the correct setting on displays; dialling a suitable concentration with one or more of dose set wheels and/or sliders, checking the correct setting on displays, so then as to: activate the fluid dosing mechanism, transfer a set substance volume from the glass vial to a separate fluid reservoir in a carrier. A user optionally checks the dose and/or concentration setting; and the method further includes inserting the dosing reservoir into the dosing module to: 1) pierce the septum(s) in the dosing reservoir(s) to establish a fluidic path from the fluid reservoir(s) in the dosing module to the fluid reservoir in the dosing reservoir; 2) pushing a button to transfer a set substance volume from the reservoir(s) in the dosing module to the dosing reservoir in the injector carrier; removing a filled dosing reservoir from the dosing module and setting aside the dosing module; inserting the filled dosing reservoir into the injector carrier to create an injector carrier assembly; moving the injector carrier assembly to a desired injection site on an animal; stroking the injection site with the injector carrier assembly to: 1) calm the animal, and 2) assess the best injection site location based on the animal's behaviour; when the animal is calm, holding the animal with one hand and with the other hand pressing an activation button on the injector to: I) insert the needle into the animal's skin, 2) deliver the proper substance dose, and, 3) retract the needle into the injector for safe disposal of the injector carrier assembly; removing the used reservoir from the injector assembly and discarding the used reservoir in a sharps container and/or keep carrier assembly for to a subsequent injection.

### BRIEF DESCRIPTION OF THE DRAWINGS

FIG. 1 is a schematic representation of preferred injection sites for an animal.

FIG. 2 is a schematic view of the insulin injection system of the invention.

FIG. 3 is a flow chart of the functionality and use of the system illustrated in FIG. 2.

FIG. 4 is a schematic view of a variant of the embodiment of FIG. 2.

FIG. 5 is a schematic view of modules/sub-systems used in the invention.

FIG. 6A is a schematic view of the disposable injector of the invention.

FIG. 6B is a second schematic view of the disposable injector of the invention.

FIG. 6C is a third schematic of the injection process used in the invention.

FIG. 7 is a schematic view of the disposable injector of the invention.

FIG. 8 is a flow chart of the use of the modules of FIG. 5 in a method of use of the invention.

FIG. 9 is a side view of the dosing module of FIG. 5.

FIG. 10 is a side perspective view of the dosing module of FIG. 7.

FIG. 11 is a side plan view of the dosing module of FIG. 5

FIG. 12 is a top view of the injector carrier and injector module of FIG. 5.

FIG. 13 is a profile view of the injector carrier and injector module of FIG. 5.

FIG. 14 is a bottom view of the injector carrier and injector module of FIG. 5.

FIG. 15 is a perspective view of the injector carrier and injector module of FIG. 5.

FIG. 16 is a disassembled view of the injector carrier and injector module of FIGS. 12-15.

FIG. 17 is an assembled perspective view of the injector carrier and injector module of

FIG. 12 and the dosing module of FIG. 11.

FIG. 18 is a flow chart of the use of the insulin delivery system.

FIG. 19 is an explanation of the use steps of the flow chart of FIG. 18.

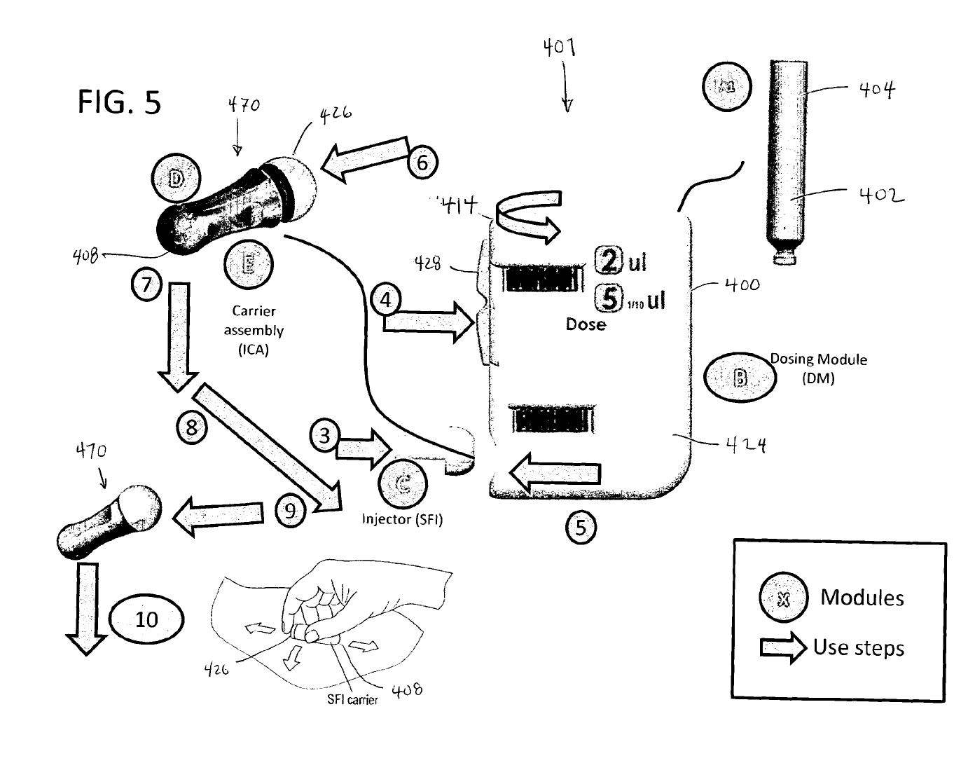

FIG. 20 is a perspective view of the dosing module of FIG. 5.

FIG. 21A is a side view of the dosing module of FIG. 5.

FIG. 21B is a side perspective view of the dosing module of FIG. 21A.

FIG. 21C is a side perspective view of the dosing module of FIG. 21A.

FIG. 22 is a view of step 1 in the use of a variant of the substance delivery system.

FIG. 23A is a view of step 2 in the use of a variant of the substance delivery system.

FIG. 23B is a view of step 2 in the use of a variant of the substance delivery system.

FIG. 24 is a view of step 3 in the use of a variant of the substance delivery system.

FIG. 25 is a view of step 4 in the use of a variant of the substance delivery system.

FIG. 26 is a view of step 5 in the use of a variant of the substance delivery system.

FIG. 27 is a view of step 6 in the use of a variant of the substance delivery system.

FIG. 28 is a view of step 7 in the use of a variant of the substance delivery system.

FIG. 29 is a view of step 8 in the use of a variant of the substance delivery system.

FIG. 30 is a view of step 9 in the use of a variant of the substance delivery system.

FIG. 31 is a view of step 10 in the use of a variant of the substance delivery system.

FIG. 32 is a flow chart of the use of the substance delivery system.

FIG. 33 is an explanation of the use steps of the flow chart of FIG. 32.

Those skilled in the art will appreciate that elements in the figures are illustrated for simplicity and clarity and have not necessarily been drawn to scale. For example, dimensions may be exaggerated relative to other elements to help improve understanding of the invention and its embodiments. Furthermore, when the terms ‘first’, ‘second’, and the like are used herein, their use is intended for distinguishing between similar elements and not necessarily for describing a sequential or chronological order. Moreover, relative terms like ‘front’, ‘back’, ‘top’ and ‘bottom’, and the like in the description and/or in the claims are not necessarily used for describing exclusive relative position. Those skilled in the art will therefore understand that such terms may be interchangeable with other terms, and that the embodiments described herein are capable of operating in other orientations than those explicitly illustrated or otherwise described.

### DETAILED DESCRIPTION OF THE PREFERRED EMBODIMENT(S)

The following description is not intended to limit the scope of the invention in any way as they are exemplary in nature and serve to describe the best mode of the invention known to the inventors as of the filing date hereof. Consequently, changes may be made in the arrangement and/or function of any of the elements described in the disclosed exemplary embodiments without departing from the spirit and scope of the invention.

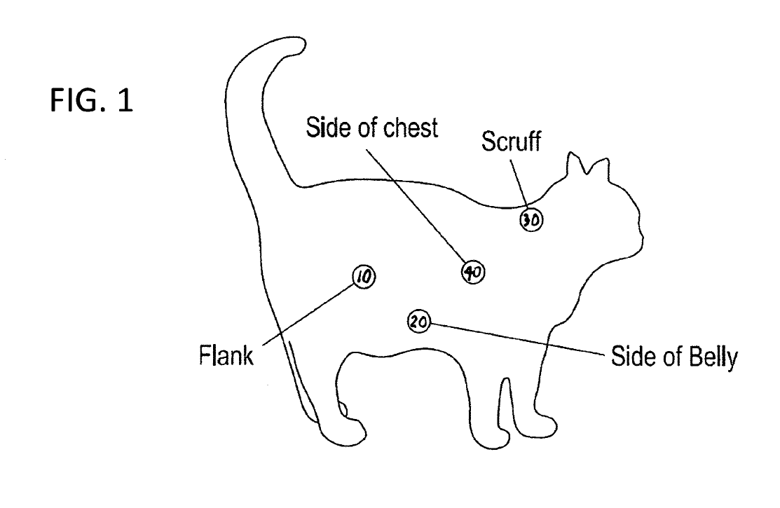

Referring now to FIG. 1, in schematic view, the injection sites for use of the system(s) of the present invention and modules thereof are provided. These exemplary injection sites include the flank 10, the side of the belly 20, the scruff 30 or the side of the chest 40. It is appreciated that since each animal exhibits different behavior, it is advisable to choose the area of the animal that is most agreeable to the animal, and it is an object of the invention to facilitate and sooth the animal prior to injection.

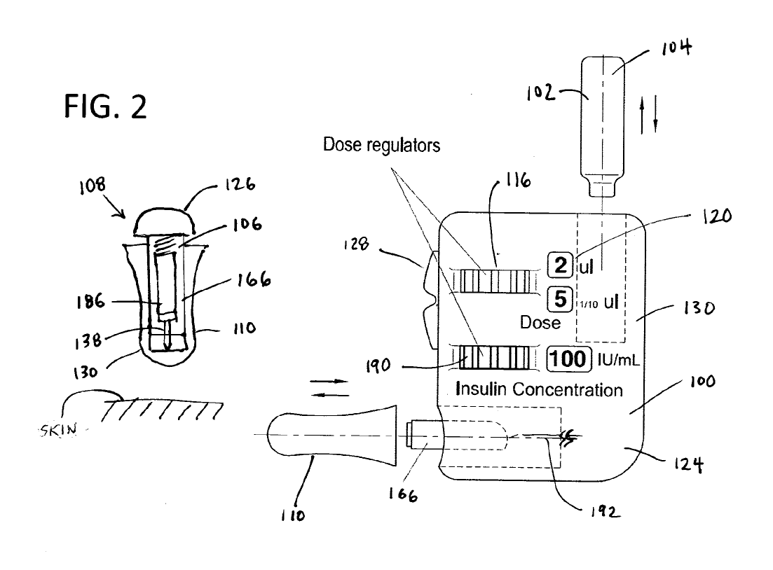

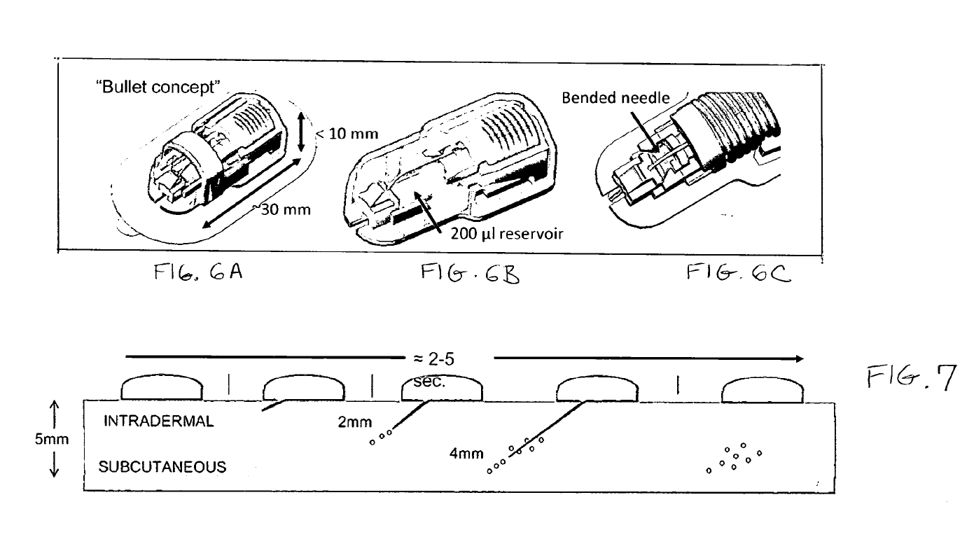

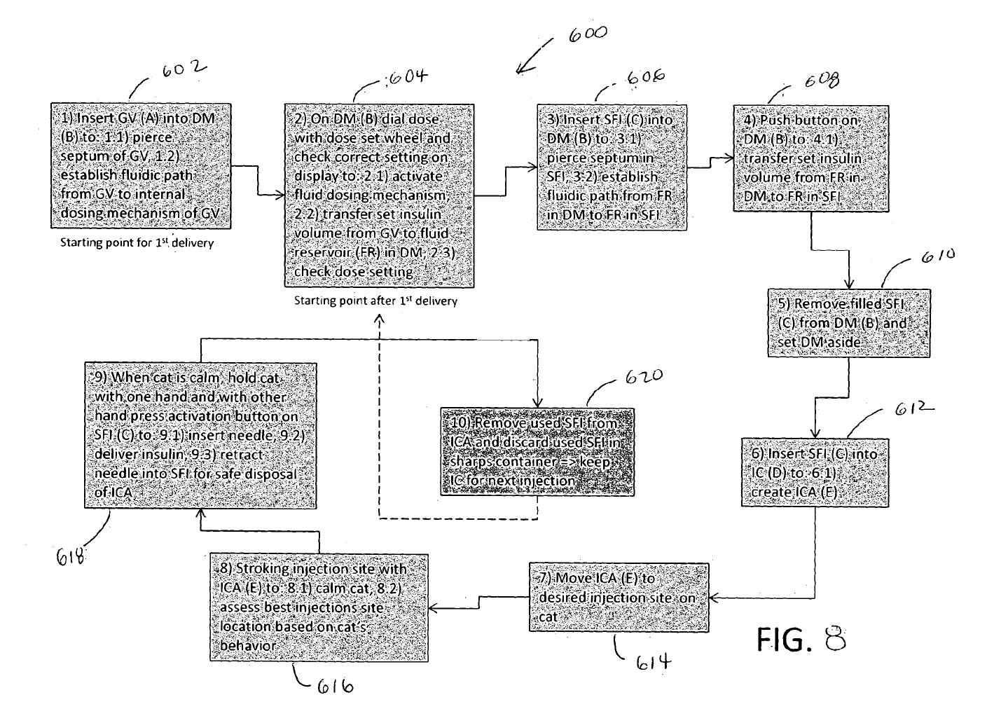

Referring now to FIG. 2, a substance delivery system 101 for dispensing a measured user selected amount of insulin, a derivate thereof, or another substance 102 into an animal is provided. While the invention, discusses its use in animal applications, it is envisioned that other variants of the invention may also be used in other mammal applications. The system 101 provides a reusable dosing module 100. The reusable dosing module 100 is adapted to extract a set volume (variably set by a user using the system based upon the mammals dosing requirements) of the insulin, a derivate thereof, or another substance 102 from a larger reservoir 104. The insulin, a derivate thereof, or another substance are made using conventional or recombination techniques and are species specific and produce the desired effects in a particular species at appropriately dosed therapeutic levels. The reusable dosing module 100 is further adapted to extract a set volume of the diluent 180 out of a reservoir 182. The reservoir 104 and/or reservoir 182 optionally includes a vial component, and the method includes transfer of the set (or variably selected) volume(s) to a single use, manually activated injector reservoir 166. The injector reservoir 166 is adapted in form from any one of the injectors described in PCT/US2012/048044, filed 25 Jul. 2012, the content of which is incorporated by reference hereto and relied upon. A suitable injector is shown in FIG. 6A. Other injector reservoir 166 forms of course may be used without departing from the spirit and scope of the invention.

A reusable injector carrier 108 is configured to house the single use manually activated injector reservoir 166 in reservoir housing cavity 186, and configured to optionally provide a surface 110 to stroke or calm the animal prior to injection. The surface 110 on the carrier 108 is textured in one variant, has soft bristles in another variant, and is of a shape and size so that the animal's behavior can be calmed. This is particularly important with sensitive animals and animals that have not grown accustomed to periodic injections. A carrier assembly housing 124 is adapted for removably housing the single use manually activated injector reservoir 166. The system 101 provides for proper dosing of the measured amount of insulin, a derivate thereof, or another substance subcutaneously into an animal.

The re-useable injector carrier 108 is adapted to trigger use of the single use manually activated injector reservoir 166 and has dampening material in the housing to dampen noise of the activation upon injection into the animal.

The carrier assembly 124 comprises a double wheel and/or slider mechanism 114 which includes a first wheel or slider mechanism 116 and a second wheel or slider mechanism 118 in one variant of the invention. In another variant, a single wheel or slider mechanism is used. The second wheel or slider mechanism 118 is adapted to regulate the dispensing of an amount of substance into the single use, manually activated injector reservoir 106. The first wheel or slider mechanism 116 is adapted to regulate the dispensing of an amount of diluent into the single use, manually activated injector reservoir 106. The use of mechanisms 116, 118, both internal and external components provides for user selection of the proper therapeutic dose of the insulin, a derivate thereof, or another substance to be transferred from vial 104 to component 166.

A first indicator 120 is adapted to indicate a first volume of insulin, a derivate thereof, or another substance 102 transferred into the single use manually activated injector reservoir 106, and a second indicator 122 that is adapted to indicate the concentration of insulin, a derivate thereof, or another substance 102 transferred into the single use manually activated injector reservoir 166. The single use manually activated injector reservoir 166 is releasably connected to the reusable dosing module 100, and snaps into or fits into cavity 190. Reservoir 166 includes a septum that is punctured by needle 192 to fill reservoir 166. In a variant, the single use, manually activated injector reservoir 166 is in measured, selected volume, fluid communication with the larger reservoir 104, through the housing 124 of reusable dosing module 100. A push button 124 is manually activated to transfer the insulin, a derivate thereof, or another substance 102 through reusable dosing module 100 into manually activated injector reservoir 166.

Importantly, the injector, to be disposable and producible in large quantities, should be standardized in terms of the volume of fluid that it injects. Consequently, preferably, the reservoir of the disposable injector has a fixed volume. One must then control the concentration of the drug or agent to be injected in order to control the dosage administered. This is the purpose of the dosage module 400. To accomplish this, the user must provide the dosing module 400 with two important inputs. One is the concentration of the drug in the drug vial (e.g. for insulin 40 IU-implied by 10 ml vial). The other is the dose of drug he wishes to provide via the micro-injector (for insulin, for example 0.5 IU). It is considered that dosing module 400 is adapted to a certain type of injector vial drug and diluent vial. Thus, in this case, dilution is carried out during each filling of the reservoir of the disposable micro-injector. Optionally, the energy required to make the dilution comes from the act of actuating the key or button on the dosing module. In doing so, the dosing module must extract a certain amount of drug in the drug vial and a suitable quantity of diluent from the diluent vial, mixing and injecting this mixture into the reservoir of the disposable injector. In this way, the amount of liquid in the injector reservoir is constant but what varies is the amount of drugs, according to dilution schedule. The reservoir of constant size (0.2 ml) is may be manufactured with a non-injectable amorphous filling. The drug, for example, insulin dose is defined by the remaining free volume in the reservoir. The insulin (veterinary standards 40-U) is not diluted

In yet a further variant, the reusable injector carrier module 108 further comprises a manual activation button and mechanism 126. The larger reservoir 104 or vial fits snugly, but removably into an internal cavity 130 within the interior of housing 124, e.g. without a friction fit or with a friction fit. The single use dosing injector 106, a part of module 108, further comprises a dosing reservoir 166. Reservoir 166 is generally much smaller in volume than vial 104, and is optionally, smaller than an optional internal reservoir in dosing module 100, which is in fluidic connection with reservoir 104 and dosing reservoir 166. In an alternate, and indeed, preferred variant, the internal reservoir of the dosing module 100 is eliminated and a direct fluidic connection (with an optional mechanical or electrical pump inserted) in line with the fluidic connection is used to transfer insulin from reservoir 104 to dosing reservoir 166 and includes one or more check valves.

The single use dosing injector 106 further comprises a retractable needle guard 130. The injector 106 comprises a needle 138 to puncture the skin of the animal to deliver the insulin.

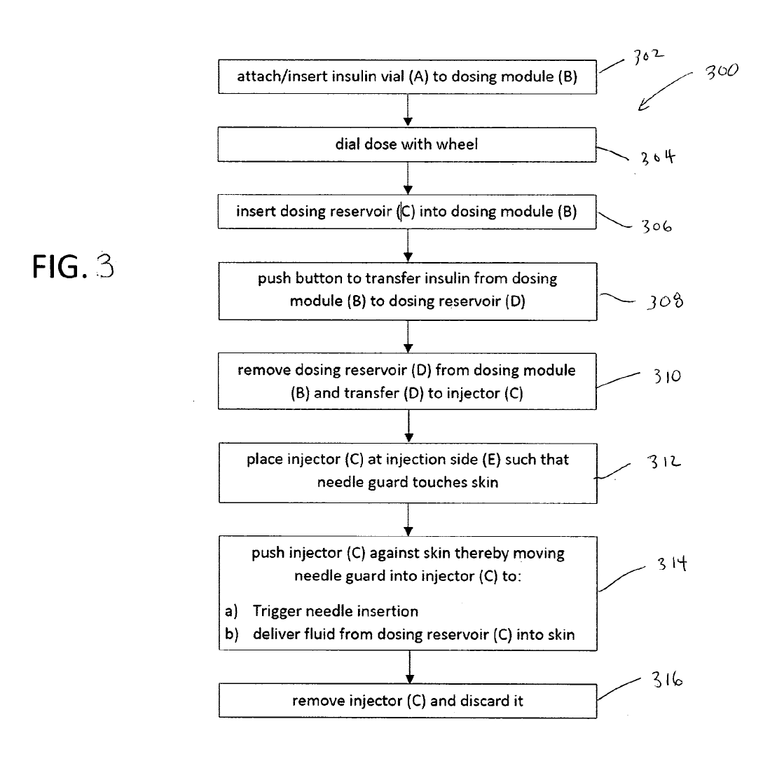

Referring now to FIG. 3, the invention provides a method 300 of dosing an animal with a variably adjustable amount of insulin, a derivate thereof, or another substance 104 and a variably adjustable amount of diluent. The method includes, one or more of the following steps, alone or in combination:
a. at step 302, inserting the glass vial 104 and/or the diluent vial 182 into dosing module 100;
b. at step 304, dialing a suitable dose of insulin, a derivate thereof, or another substance 102 and/or a suitable amount of diluent with wheel or slider 116 and/or wheel or slider 118;
c. at step 306, inserting dosing reservoir 166 into dosing module 100;
d. at step 308, pushing button 128 to transfer substance 102 from dosing module 100 to dosing reservoir 166;
e. at step 310, removing dosing reservoir 166 from dosing module 100 and transferring the dosing reservoir 166 to injector 106;
f. at step 312, placing injector 106 at injection site 140 such that the needle guard 130 touches the skin of the animal;
g. at step 314, pushing injector 106 against the skin of the animal thereby moving retractable needle guard 130 up and into injector 106 to 1) trigger needle 138 insertion through the skin of the animal and 2) dispense the insulin, a derivate thereof, or another substance 102 from dosing reservoir 166 into the skin of the animal; and,
h. at step 316, removing the injector 106 and discarding it.

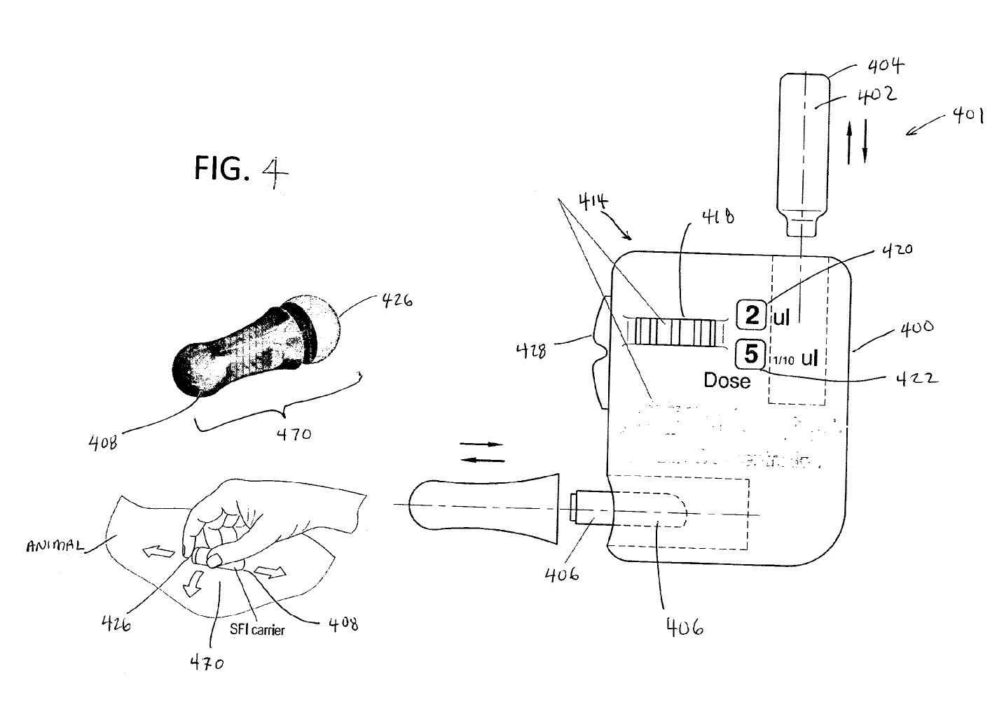

Referring now to FIG. 4, the invention provides an insulin delivery system 401 for dispensing a measured amount of insulin or a derivative thereof 402 into an animal which includes a number of modules (FIG. 5). The system 401 includes a reusable dosing module 400. The reusable dosing module 400 is adapted to extract a set volume of the insulin, a derivate thereof, or another substance 402 from a reservoir 404 and/or to extract a set volume of diluent 452 from a reservoir 454. The reservoir 404 and/or reservoir 454 optionally comprises a vial (e.g. a glass vial), and transfers the set volume(s) to a single use, manually activated injector reservoir 406 of carrier assembly 470. The reusable injector carrier assembly 470 is configured to house the single use manually activated injector reservoir 406 and configured to provide a surface 408 to stroke or calm the animal prior to injection as described above, on the exterior housing of the carrier.

A micro-injector pre-filled with insulin is hosted in an injector carrier for the injection. The micro-injector is self-powered by two springs: one for needle penetration and insulin infusion and the other one for needle retraction. The injection is triggered at the push of a button on the micro-injector/carrier when the animal is calm.

A carrier assembly 424 is adapted for removably housing the single use manually activated injector assembly 470, such that the system 401 provides for proper dosing of the measured amount of fluid subcutaneously into an animal via the assembly 470. The re-useable injector carrier assembly 470 is adapted to trigger use of the single use manually activated injector reservoir 406 insulin volume, and to dampen noise of the activation upon injection into the animal.

The carrier assembly 442 comprises a double wheel and/or slider mechanism 414 including a first wheel or slider mechanism 416 and a second wheel or slider mechanism 418. The second wheel or slider mechanism 418 is adapted to dispense an amount of substance into the single use, manually activated injector 406. The first wheel or slider mechanism 416 is adapted to regulate the dispensing of an amount of diluent into the single use, manually activated injector 406. A first indicator 420 is adapted to indicate that a first volume of fluid 402 has been transferred into the single use manually activated injector 406, and a second indicator 422 is adapted to indicate a second volume of insulin 402 has been transferred into the single use manually activated injector 406, the first volume being greater than the second volume. Generally, micro-liter volumes and 1/10 of microliter volumes are indicated, but of course any suitable volume indicator can also be used with the invention. A third indicator 456 is adapted to indicate the substance concentration. The single use manually activated injector assembly 470 is releasably connected to the reusable dosing module 400. The single use, manually activated injector reservoir 406 is in measured fluid communication with the reservoir 404 and/or the reservoir 454, through the housing 424 of reusable dosing module 400. A push button 428 is manually activated to transfer the fluid 402 through re-useable dosing module 400 into manually activated injector reservoir 406. The transfer involves a push button pump in one variant of the invention, and an electrically operated (e.g. battery) operated pump in another variant. In yet a further variant, the reusable injector carrier assembly 470 further comprises a manual activation button and mechanism 426.

Referring now to FIGS. 5, 6A, 6B, 32 and 33, various modules used in the invention and methods of using the modules are described. The method 600 and method 4100 of dosing an animal with a variably adjustable amount of fluid 402 and/or a variably adjustable amount of diluent 452 via dosing module 400 includes the steps of:
i. at step 602, inserting the glass vial 404 and/or a diluent vial 454 into dosing module 400 to:
   i. pierce the septum of the glass vial 404 and/or a diluent vial 454, and
   ii. establish a fluidic path from the glass vial 404 and/or a diluent vial 454 to the internal dosing mechanism of glass vial 404 and/or a diluent vial 454 and the internal fluidic path/mechanism of dosing module 400;
j. at step 604, dialling a suitable dose of fluid with one or more of dose set wheel(s) and/or slider(s) 416, 418, and dialling a suitable dose of diluent with a wheel or slider (optionally checking the correct setting on displays 420, 422, 456) to:
   i. activate the fluid dosing mechanism,
   ii. transfer a set substance volume from the glass vial 404 and/or a diluent vial 454 to a separate optional fluid reservoir in carrier 400, and,
   iii. (a user optionally checks the dose and/or concentration setting);
k. at step 606, inserting dosing reservoir 466 into dosing module 400 to:
   i. pierce the septum in the dosing reservoir 466 to establish a fluidic path from the fluid reservoir in the dosing module 400 for the fluid to transfer to the dosing reservoir 466;
l. at step 608, pushing button 428 to transfer a set substance 402 volume and the diluent volume 452 from the reservoir(s) in the dosing module 400 to the dosing reservoir 466 in injector carrier 408;
m. at step 610, removing filled dosing reservoir 466 from dosing module 400 and setting aside dosing module 400;
n. at step 612, inserting the filled dosing reservoir 466 into injector carrier 406 to create injector carrier assembly 470;
o. at step 614, moving injector carrier assembly 470 to desired injection site on an animal;
p. at step 616, stroking the injection site with the injector carrier assembly 470 to:
   i. calm the animal, and
   ii. assess the best injection site location based on the animal's behaviour;
q. at step 618, when the animal is calm, holding the animal typically with one hand and with the other hand pressing activation button 426 on the injector carrier assembly 470 to:
   i. insert needle,
   ii. deliver substance, and
   iii. retract needle into injector 406 for safe disposal of the injector carrier assembly 470;
r. at step 620, removing the used reservoir 466 from the injector assembly 470 and discarding the used reservoir 466 in a sharps container and/or keep carrier assembly 470 for a subsequent injection.

A progression of an injection performed using the invention includes a standby, an insertion, an infusion and a retraction stage. In the standby stage, the needle is inside the assembly 470, and the fluid reservoir is full, with the device held against the skin of the animal. In the insertion stage, the needle is inserted 1.5 mm to 2 mm beneath the skin into the intradermal skin layer. In the infusion stage, the fluidic channel is then opened and the reservoir begins to empty as the needle continues to travel to the 4 mm to 5 mm subcutaneous target depth. The flow rate depends greatly on the reservoir design (material properties, geometry, etc.), size of the fluidic channel (needle inner diameter), and resistance beneath the skin.

Optionally, an indicator of carrier assembly 470 indicates whether the injection has been completed. It is important to provide feedback so the user knows the infusion process is complete and the device is ready for removal. Alternatively, a snap type interface can be implemented, so as the spring retracts, there is a “click” when the needle holder reaches the final position. A viewing window can be implemented as well, where a portion of the needle is flagged with a color, this portion moving behind a window, indicating that the needle holder has retracted.

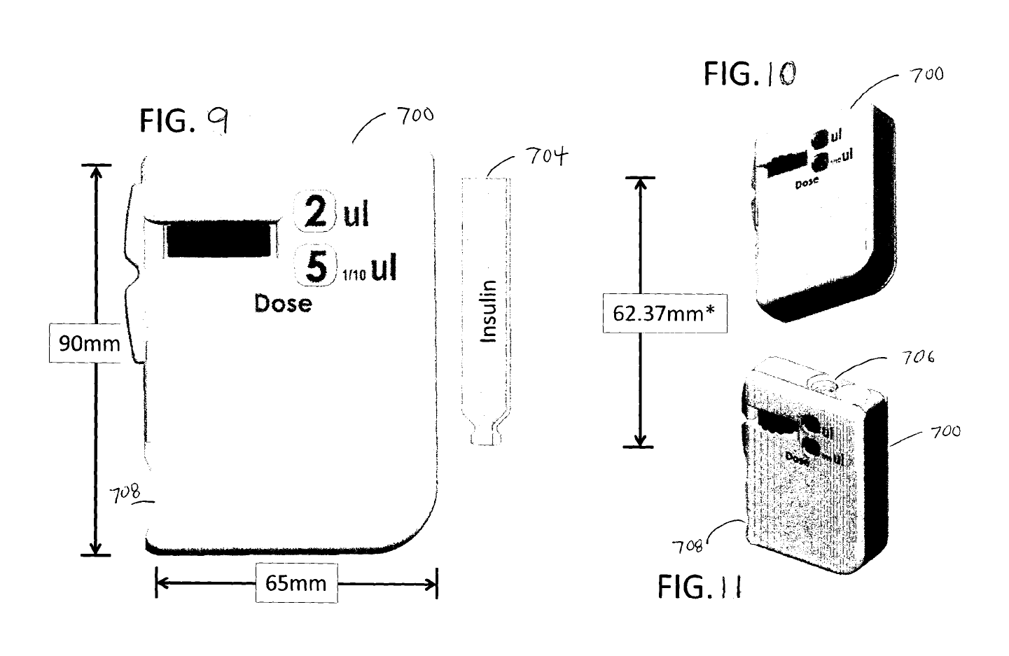

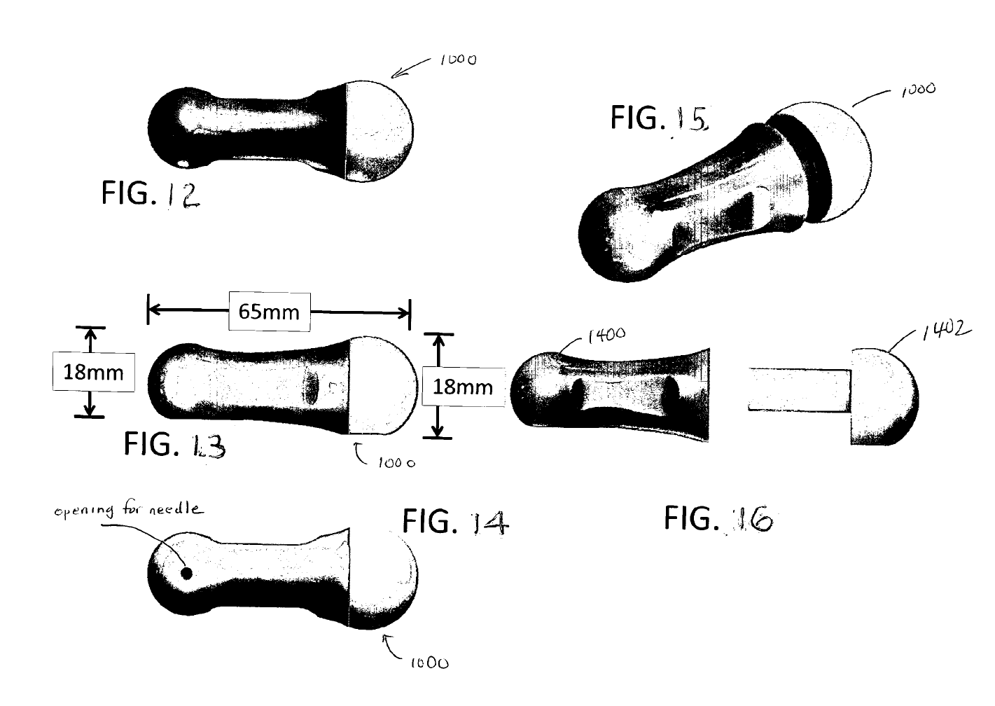

Now referring to FIGS. 9-11, a variant of the dosing module 700 of FIG. 5 is illustrated along with glass vial 704. Module 700 includes a first vial insertion cavity 706 for glass vail 704 and a second insertion cavity 708 for insertion of injector 1402 (FIG. 16) which mates with injector carrier 1400. Preferred dimensions for the dosing module 700 are also provided.

Now referring to FIGS. 12-15, different views are provided of the injector carrier and injector module 1000 as assembled. Now referring to FIG. 16, the injector carrier and injector module is illustrated in a disassembled view and includes injector carrier 1400 which mates with an injector 1402.

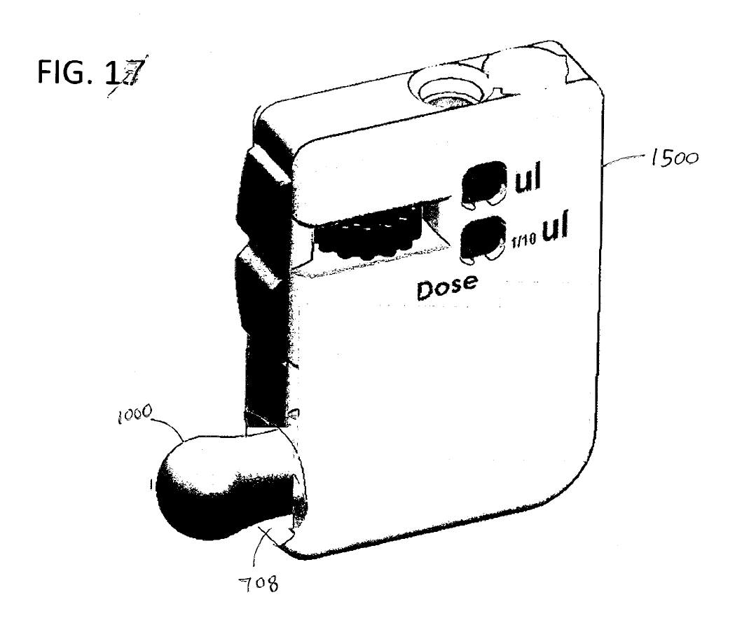

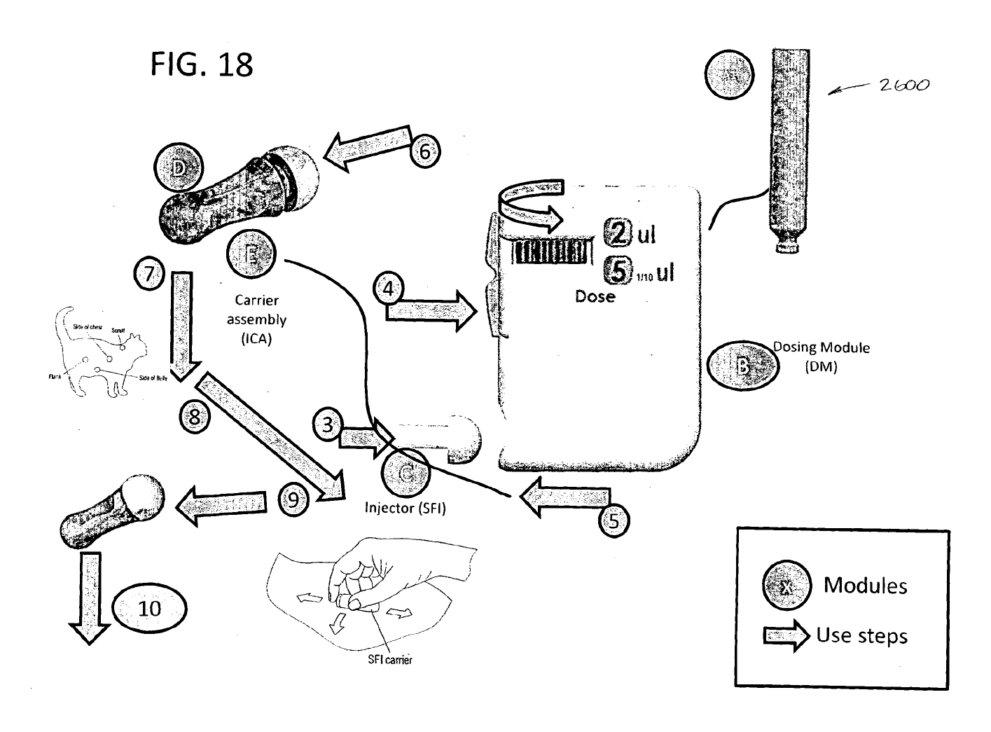

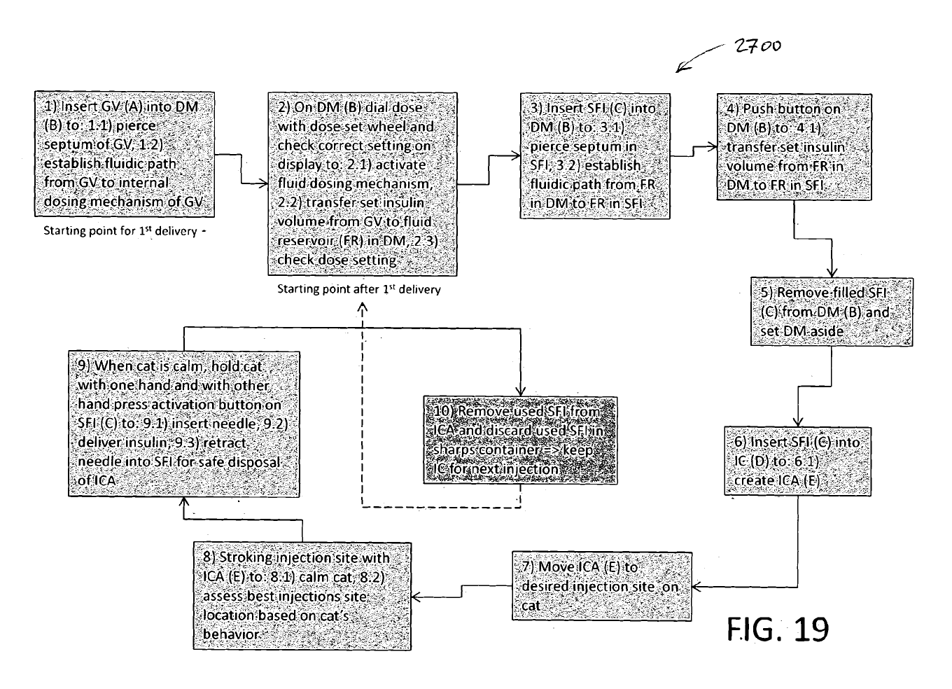

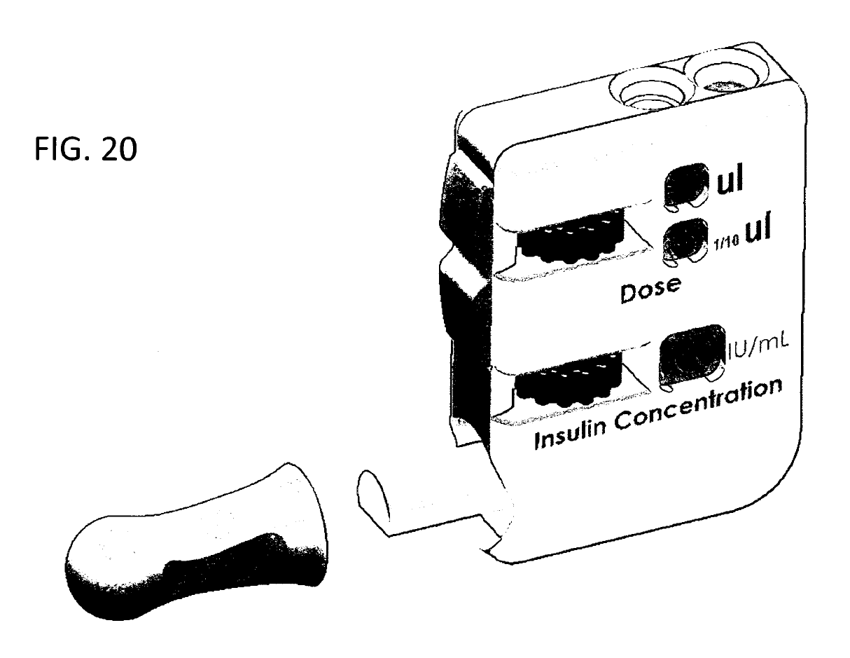

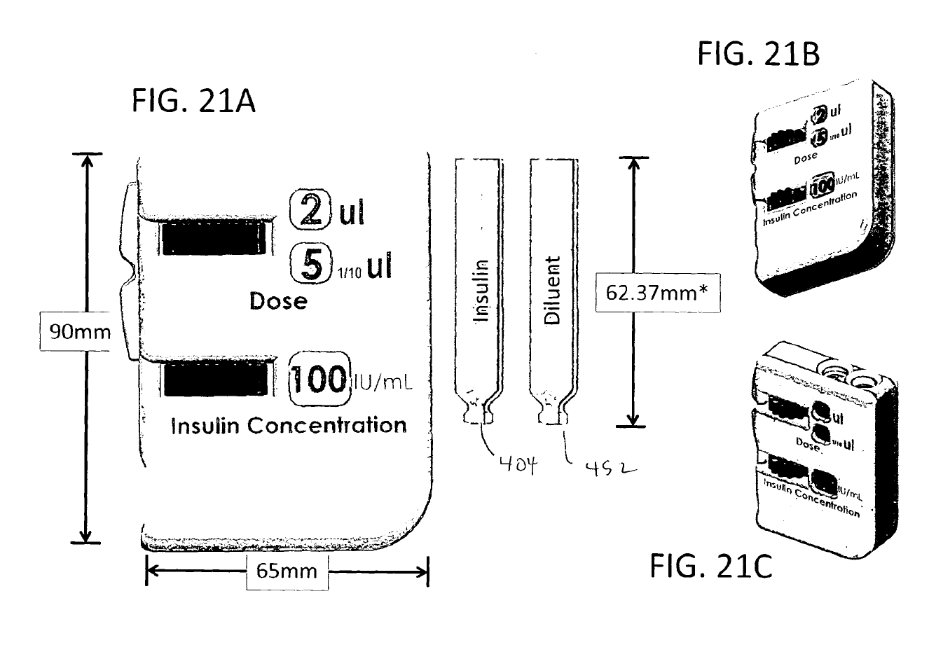

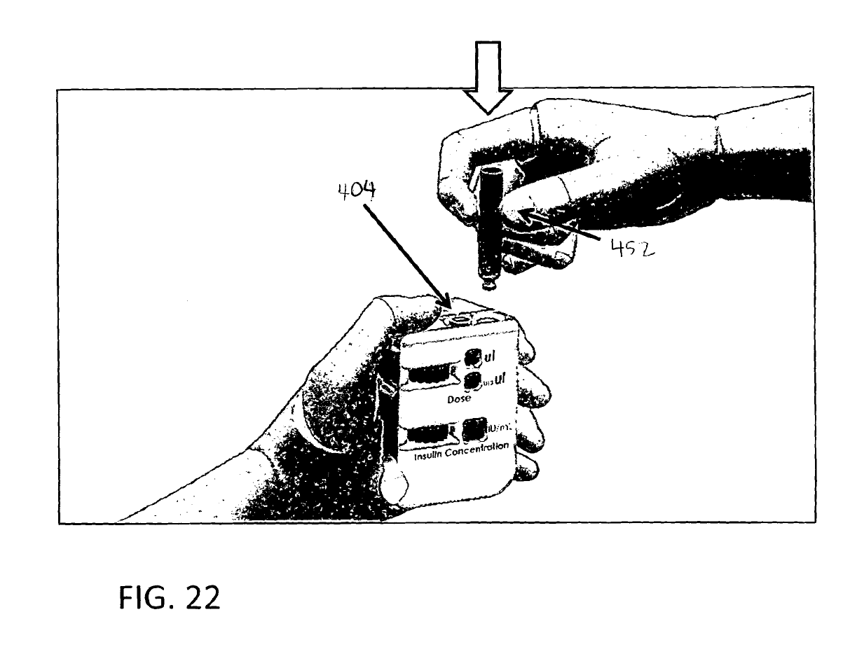

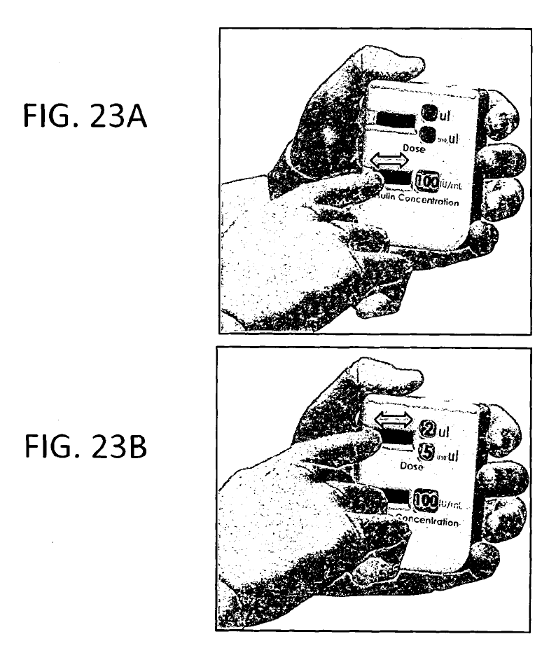

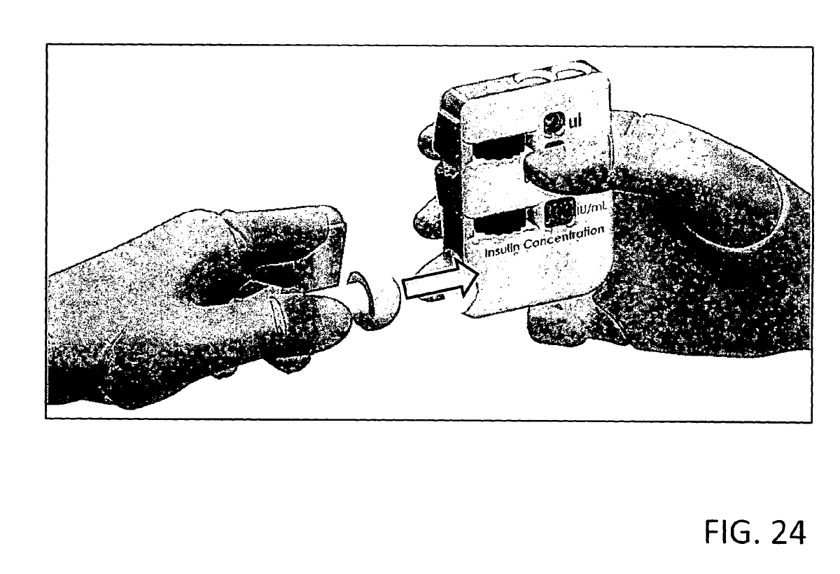

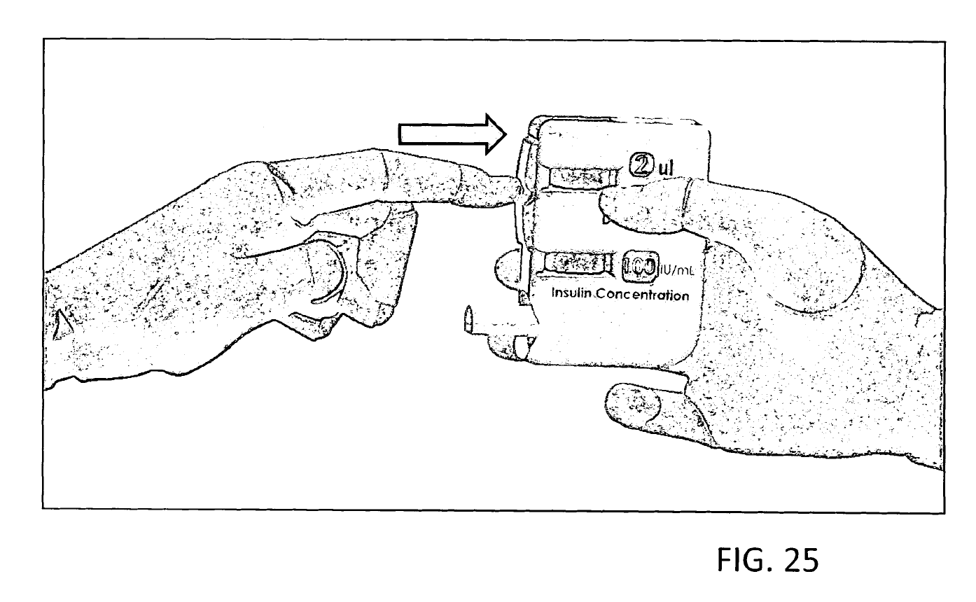

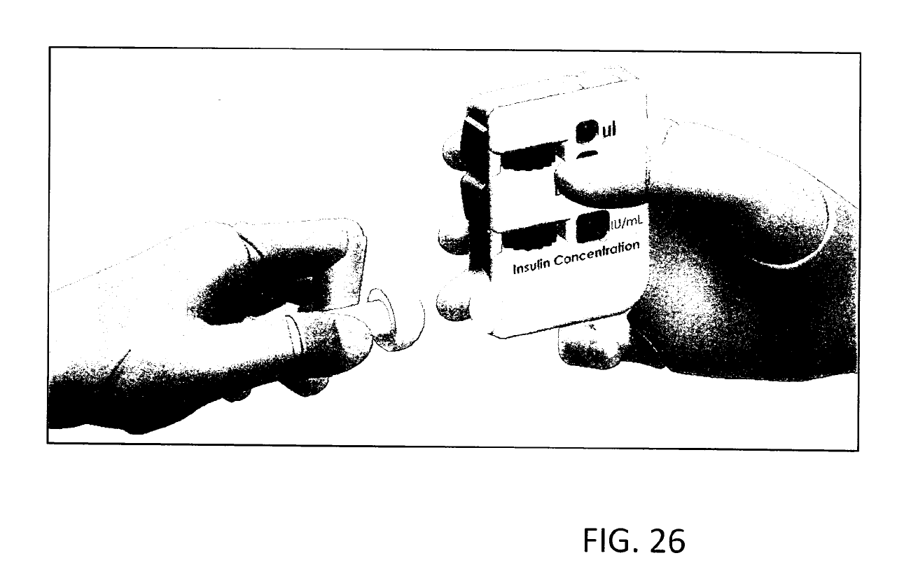

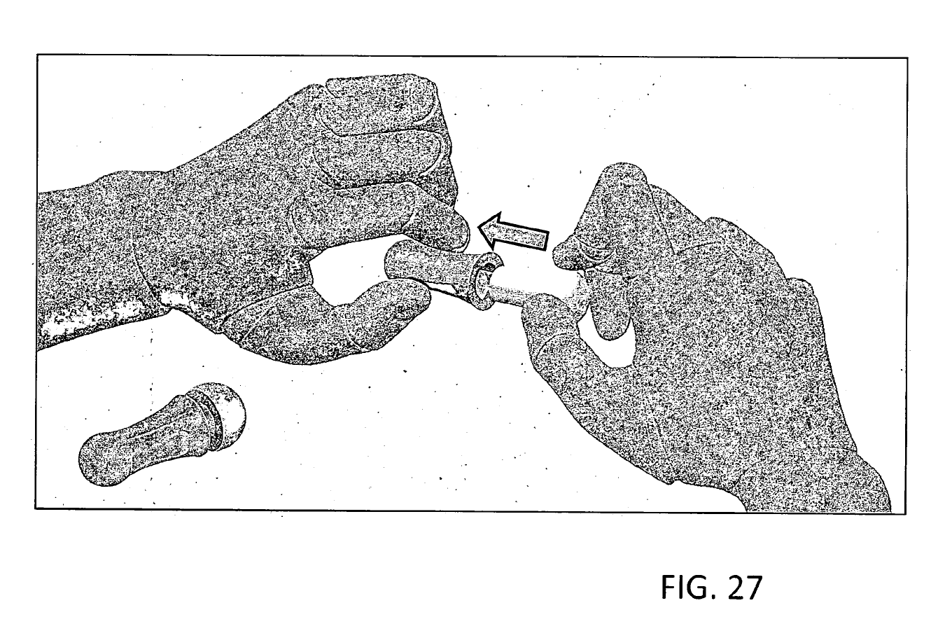

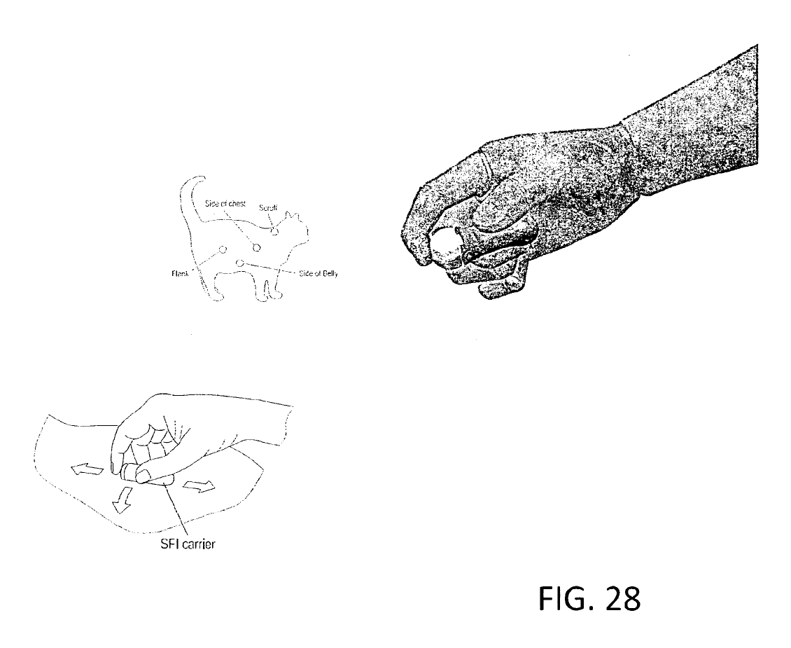

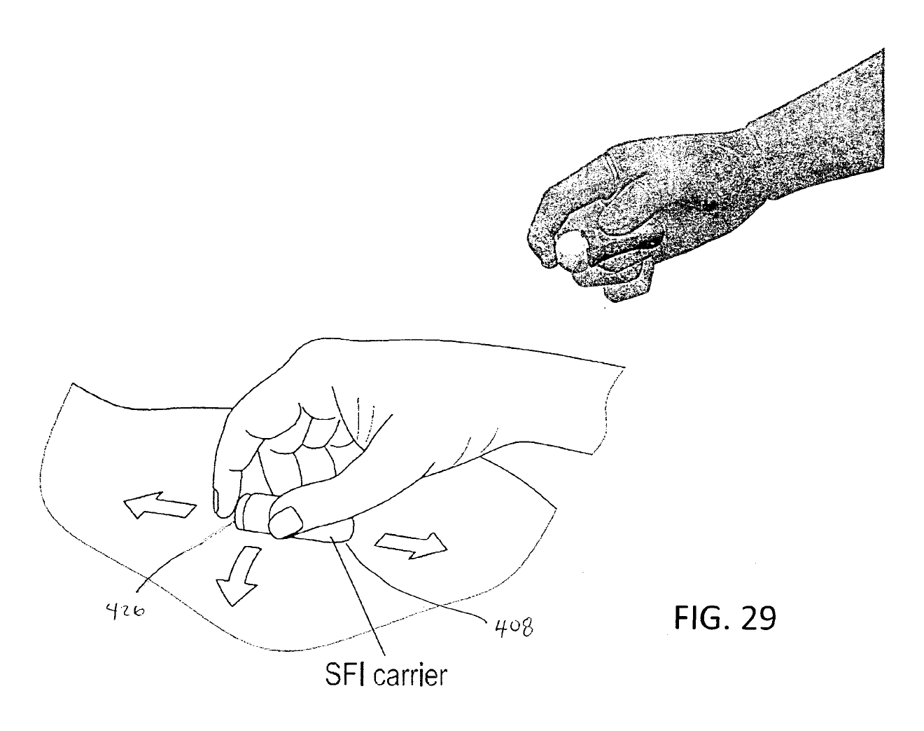

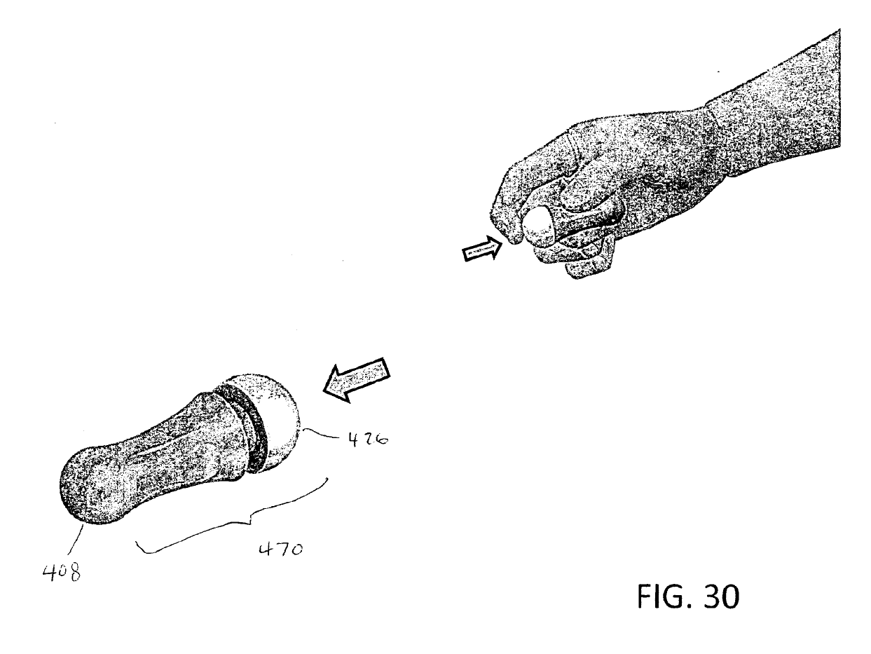

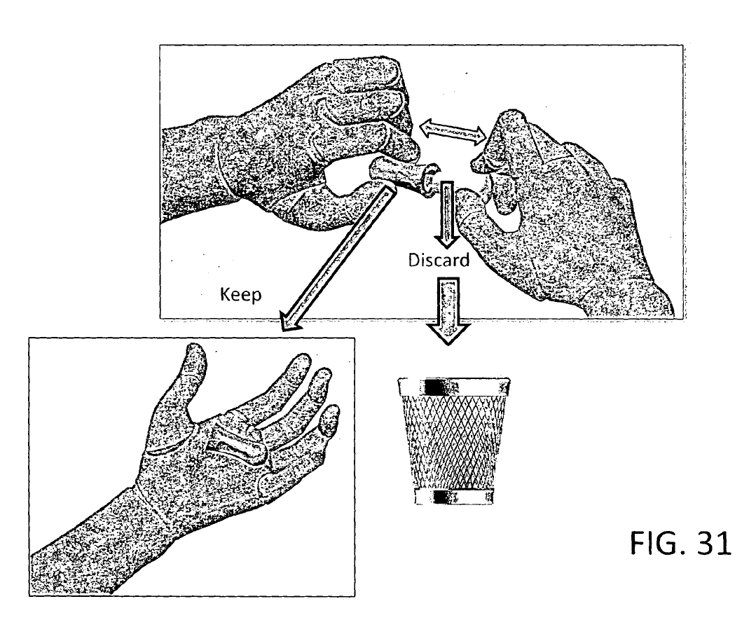

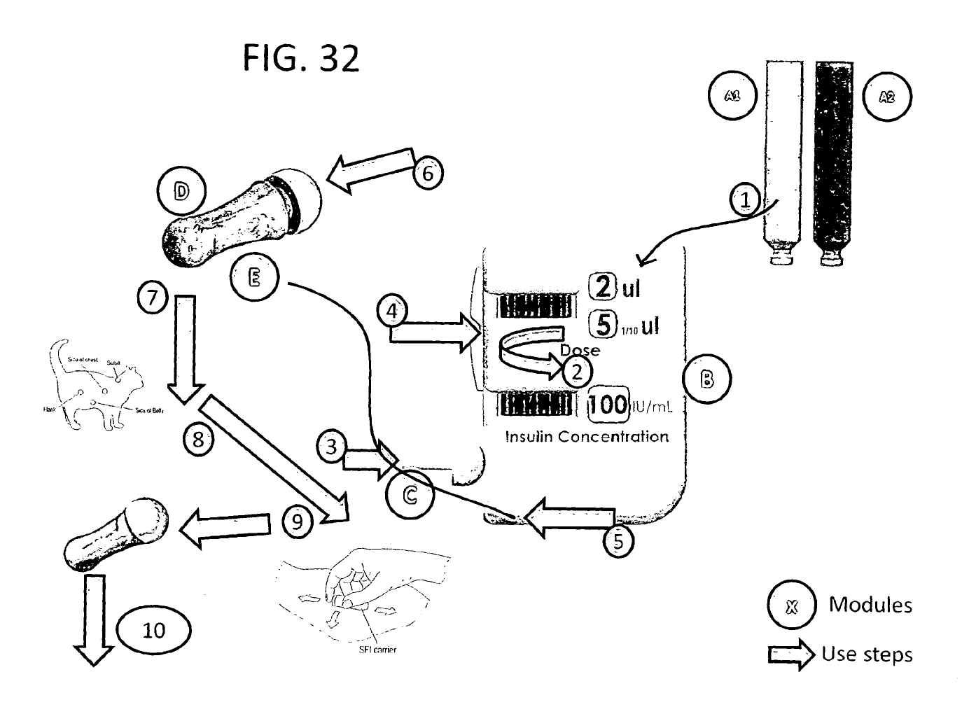

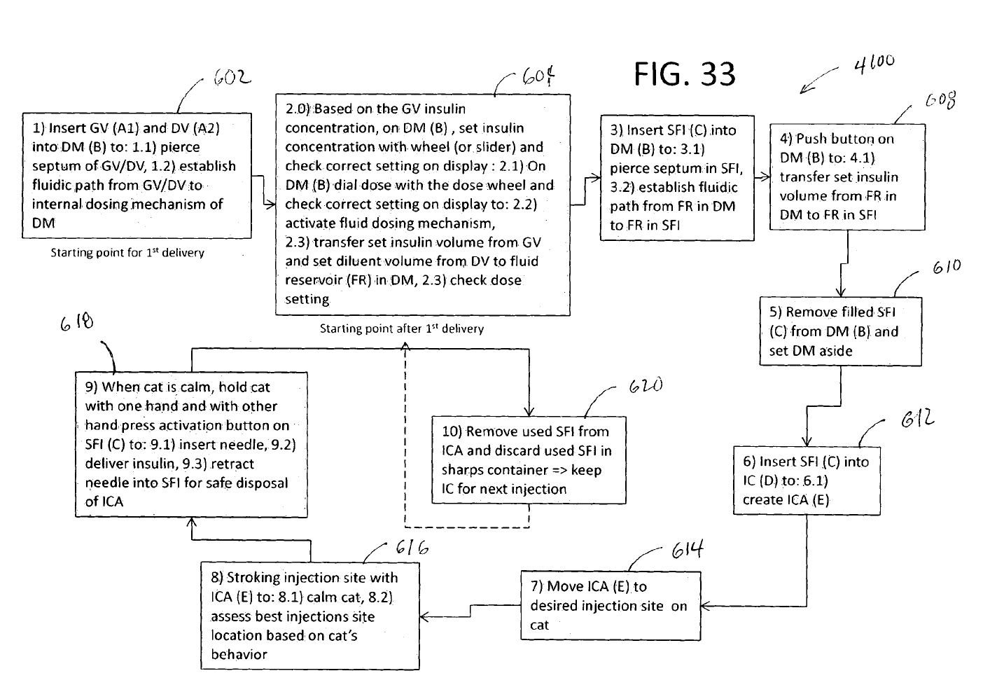

Now referring to FIG. 17, an assembled perspective view of the injector carrier and injector module 1000 and the dosing module 1500 is provided. Module 1000 fits matingly and removably in cavity 708.

Now referring to FIG. 18, method of use steps 1-10 and the various modules used in each respective method of use step are illustrated, individually, in the use of the substance delivery system.

Now referring to FIG. 18, flow chart 2600 illustrates the use of the fluid delivery system as further detailed in substance delivery system use steps explanation of FIG. 19.

Now referring to FIG. 31, method of use steps 1-10 and the various modules used in each respective method of use step are illustrated, individually, in the use of a variant of the substance delivery system.

Now referring to FIG. 32, flow chart 2600 illustrates the use of the insulin or a derivate thereof delivery system as further detailed in substance delivery system use steps explanation of FIG. 33.

It should be appreciated that the particular implementations shown and described herein are representative of the invention and its best mode and are not intended to limit the scope of the present invention in any way. Furthermore, any connecting lines shown in the various figures contained herein are intended to represent exemplary functional relationships and/or physical couplings between various elements. It should be noted that many alternative or additional physical connections or functional relationships may be present and apparent to someone of ordinary skill in the field.

Moreover, the apparatus, system and/or method contemplate the use, sale and/or distribution of any goods, services or information having similar functionality described herein.

The specification and figures are to be considered in an illustrative manner, rather than a restrictive one and all modifications described herein are intended to be included within the scope of the invention claimed, even if such is not specifically claimed at the filing of the application. Accordingly, the scope of the invention should be determined by the claims appended hereto or to later amended or added, and their legal equivalents rather than by merely the examples described above. For instance, steps recited in any method or process claims should be construed as being executable in any order and are not limited to the specific order presented in any claim. Further, the elements and/or components recited in any apparatus claims may be assembled or otherwise operationally configured in a variety of permutations to produce substantially the same result as the present invention. Consequently, the invention is not limited to the specific configuration recited in the claims.

Benefits, other advantages and solutions mentioned herein are not to be construed as necessary, critical, or essential features or components of any or all the claims.

In an advantage, the system and method of the invention allows accommodates a standard size disposable injector yet allows injections of differing dosages of drug.

In another advantage, the system and method of the invention allows for highly accurate dosing of a drug.

In another advantage, the system and method of the invention allows for safe delivery of a drug by essentially eliminating the chance of being cut or inadvertently injected by the needle of the injector.

As used herein, the terms “comprises”, “comprising”, or any variation thereof, are intended to refer to a non-exclusive listing of elements, such that any process, method, article, composition or apparatus of the invention that comprises a list of elements does not include only those elements recited, but may also include other elements described in this specification. The use of the term “consisting” or “consisting of” or “consisting essentially of” is not intended to limit the scope of the invention to the enumerated elements named thereafter, unless otherwise indicated. Other combinations and/or modifications of the above-described elements, materials or structures used in the practice of the present invention may be varied or otherwise adapted by the skilled artisan to other design without departing from the general principles of the invention.

The patents and articles mentioned above are hereby incorporated by reference herein, unless otherwise noted, to the extent that the same are not inconsistent with this disclosure.

Other characteristics and modes of execution of the invention are described in the appended claims.

Further, the invention should be considered as comprising all possible combinations of every feature described in the instant specification, appended claims, and/or drawing figures which may be considered new, inventive and industrially applicable.

Multiple variations and modifications are possible in the embodiments of the invention described here. Although certain illustrative embodiments of the invention have been shown and described here, a wide range of modifications, changes, and substitutions is contemplated in the foregoing disclosure. While the above description contains many specifics, these should not be construed as limitations on the scope of the invention, but rather as exemplifications of one or another preferred embodiment thereof. In some instances, some features of the present invention may be employed without a corresponding use of the other features. In addition, the term “flexible” as used herein encompasses the concept of variable, in that a variable volume reservoir should be considered a flexible chamber, even if no individual components flex. Accordingly, it is appropriate that the foregoing description be construed broadly and understood as being given by way of illustration and example only, the spirit and scope of the invention being limited only by the claims which ultimately issue in this application.

1. A fluid delivery system for dispensing a measured, pre-selected amount of a drug to a target, comprising:
(a) a reusable dosing module the reusable dosing module being adapted to extract a set amount of the drug from a first reservoir, the first reservoir optionally comprising a first vial, and being adapted to extract a set volume of diluent from a second reservoir, the second reservoir optionally comprising a second vial, and transfer the selected, set volume(s) to a single use, manually activated injector reservoir, the injector reservoir being removably housed in a cavity of a housing of the reusable dosing module, and
(b) a reusable injector carrier assembly configured to house the single use manually activated injector reservoir and configured to provide a smooth surface to stroke or calm an animal target prior to fluid delivery, and a cavity adapted for removably housing the single use manually activated injector reservoir,
whereby the system is adapted to provide for user selected therapeutic dosing of the measured amount of the fluid and user selected amount of diluent.

2. The fluid delivery system of claim 1, wherein the target is an animal, and the dispensing of the fluid is intended to be performed subcutaneously into the target animal.

3. A single use manually activated injector reservoir adapted to be used with the fluid delivery system of claim 1, wherein the injector reservoir this adapted to be charged with fluid by the dosing module.

4. A single use manually activated injector reservoir adapted to be used with the fluid delivery system of claim 1, wherein the injector reservoir is adapted to be filled with a drug.

5. A method of delivering a fluid, the method including the steps of:
a. inserting the glass vial and/or the diluent vial into the dosing module of claim 1;
b. dialling a suitable dose of insulin, a derivate thereof, or another substance and/or a suitable amount of diluent with wheel or slider and/or wheel or slider;
c. inserting injector reservoir into dosing module;
d. pushing button to transfer substance from dosing module to injector reservoir;
e. removing injector reservoir from dosing module and transferring the injector reservoir to injector assembly comprising a needle guard;
f. placing injector assembly at injection site such that a needle guard touches the skin of the animal;
g. pushing injector assembly against the skin of the animal thereby moving retractable needle guard up and into injector assembly to
i. trigger needle insertion through the skin of the animal and
ii. dispense the insulin, a derivate thereof, or another substance from dosing reservoir into the skin of the animal; and,
h. removing the injector assembly and discarding it.

6. A method of dosing an animal with a variably adjustable amount of fluid and/or a variably adjustable amount of diluent via dosing module of claim 1, the method includes the steps of:
a. inserting the glass vial and/or a diluent vial into the dosing module to:
i. pierce the septum of the glass vial and/or a diluent vial, and
ii. establish a fluidic path from the glass vial and/or a diluent vial to the internal dosing mechanism of glass vial and/or a diluent vial and the internal fluidic path/mechanism of dosing module;
b. dialling a suitable dose of fluid with one or more of dose set wheel(s) and/or slider(s), and dialling a suitable dose of diluent with a wheel or slider, optionally checking setting, to:
i. activate the fluid dosing mechanism, and
ii. transfer a set substance volume from the glass vial and/or a diluent vial to a separate optional fluid reservoir in carrier, and,
iii. inserting dosing reservoir into dosing module to pierce the septum in the dosing reservoir to establish a fluidic path from the fluid reservoir in the dosing module for the fluid to transfer to the dosing reservoir;
c. pushing button to transfer a set substance volume and the diluent volume from the reservoir(s) in the dosing module to the dosing reservoir in injector carrier;
d. removing filled dosing reservoir from dosing module and setting aside dosing module;
e. inserting the filled dosing reservoir into injector carrier to create injector carrier assembly;
f. moving injector carrier assembly to desired injection site on an animal;
g. stroking the injection site with the injector carrier assembly to:
i. calm the animal, and
ii. assess the best injection site location based on the animal's behaviour;
h. holding the animal typically with one hand and with the other hand pressing activation button on the injector carrier assembly to:
i. insert needle,
ii. deliver substance, and
iii. retract needle into injector for safe disposal of the injector carrier assembly;
i. removing the used reservoir from the injector assembly and discarding the used reservoir in a sharps container and/or keep carrier assembly for a subsequent injection.

7. A fluid delivery system for dispensing a measured, pre-selected amount of a drug to a target, comprising:
(a) a reusable dosing module, the reusable dosing module being adapted to extract a set amount of the drug from a first reservoir, the first reservoir optionally comprising a first vial, and being adapted to extract a set volume of diluent from a second reservoir, the second reservoir optionally comprising a second vial, and transfer the selected, set volume(s) to a single use, manually activated injector reservoir, the injector reservoir being removably housed in a cavity of a housing of the reusable dosing module, and
(b) a reusable injector carrier assembly configured to house the single use manually activated injector reservoir and optionally configured to provide a smooth surface to stroke or calm an animal target prior to fluid delivery, and a cavity adapted for removably housing the single use manually activated injector reservoir,
whereby the system is adapted to provide for user selected therapeutic dosing of the measured amount of the fluid and user selected amount of diluent.
